(19) United States
(12) Patent Application Publication
(10) Pub. No.: US 2020/0327985 A1
(43) Pub. Date: Oct. 15, 2020

(54) MULTIMODAL FRAMEWORK FOR HEART ABNORMALITIES ANALYSIS BASED ON EMR/EHR AND ELECTROCARDIOGRAPHY

(71) Applicant: TENCENT AMERICA LLC, Palo Alto, CA (US)

(72) Inventors: Nan DU, Santa Clara, CA (US); Kun Wang, San Jose, CA (US); Min Tu, Cupertino, CA (US); Shangqing Zhang, San Jose, CA (US); Hui Tang, Mountain View, CA (US); Shih-Yao Lin, Palo Alto, CA (US); Wei Fan, New York, NY (US)

(73) Assignee: TENCENT AMERICA LLC, Palo Alto, CA (US)

(21) Appl. No.: 16/378,772

(22) Filed: Apr. 9, 2019

(51) Int. Cl. G16H 50/20

(52) U.S. Cl. CPC G16H 50/20; G06N 20/10

(57) ABSTRACT

A method of performing a heart abnormalities analysis, includes learning text information from an electronic medical record (EMR) and/or an electronic health record (EHR) of a user, learning signal information from electrocardiography (ECG) signal data of the user, merging the learned text information and the learned signal information to generate one or more representations of the text information and the signal information that are merged, and performing the heart abnormalities analysis on the generated one or more representations.

### BACKGROUND

An electrocardiography (ECG) exam is one of the most common medical procedures that can help doctors diagnose many heart diseases, including atrial fibrillation, myocardial infarction, and acute coronary syndrome (ACS). Annually, around 300 million ECGs are recorded. Conventional approaches for ECG analysis tend to use digital signal processing algorithms, such as wavelet transformations, to compute features from ECG signals. Recently, more and more approaches adopt deep neural networks, such as a convolutional neural network (CNN) and a recurrent neural network (RNN), and achieve good accuracy for multi-class classification tasks based on ECG signals. However, most of the existing works can only work on the electric signal information, which cannot provide comprehensive information on a patient's health status regardless of a patient's electronic medical record (EMR) or electronic health record (EHR).

### SUMMARY

According to embodiments, a method of performing a heart abnormalities analysis, includes learning text information from an electronic medical record (EMR) and/or an electronic health record (EHR) of a user, learning signal information from electrocardiography (ECG) signal data of the user, merging the learned text information and the learned signal information to generate one or more representations of the text information and the signal information that are merged, and performing the heart abnormalities analysis on the generated one or more representations.

According to embodiments, an apparatus for performing a heart abnormalities analysis, includes at least one memory configured to store program code, and at least one processor configured to read the program code and operate as instructed by the program code. The program code includes first learning code configured to cause the at least one processor to learn text information from an electronic medical record (EMR) and/or an electronic health record (EHR) of a user, second learning code configured to cause the at least one processor to learn signal information from electrocardiography (ECG) signal data of the user, merging code configured to cause the at least one processor to merge the learned text information and the learned signal information to generate a representation of the text information and the signal information that are merged, and performing code configured to cause the at least one processor to perform the heart abnormalities analysis on the generated representation.

According to embodiments, a non-transitory computer-readable medium storing instructions that, when executed by at least one processor of a device, cause the at least one processor to learn text information from an electronic medical record (EMR) and/or an electronic health record (EHR) of a user, learn signal information from electrocardiography (ECG) signal data of the user, merge the learned text information and the learned signal information to generate a representation of the text information and the signal information that are merged, and perform a heart abnormalities analysis on the generated representation.

### BRIEF DESCRIPTION OF THE DRAWINGS

FIG. 1 is a diagram of an environment in which methods, apparatuses and systems described herein may be implemented, according to embodiments.

FIG. 2 is a diagram of example components of one or more devices of FIG. 1.

FIG. 3 is a diagram of a multimodal framework for heart abnormalities analysis based on an EMR and/or an EHR and ECG signal data of a patient, according to embodiments.

FIG. 4 is a flowchart of a method of performing a heart abnormalities analysis, according to embodiments.

FIG. 5 is a diagram of an apparatus for performing a heart abnormalities analysis, according to embodiments.

### DETAILED DESCRIPTION

Multimodal Heart Abnormalities Analysis (MHAA) is a new framework for training analytical models with both patient EMR/EHR data in text and ECG data in signal. MHAA can be applied widely, in ECG classification, computer-aided diagnosis, bedside alarms and patient ECG monitoring.

A standard ECG report contains signals from 12 different leads that requires 10 electrodes in contact with a body. These electrodes are located on different specific locations of the body. With such geometric placements, ECG can measure and trace electrophysiologic patterns during each heartbeat. Further, the electrical changes collected from the electrodes are used to derive waveform signals on multiple axes.

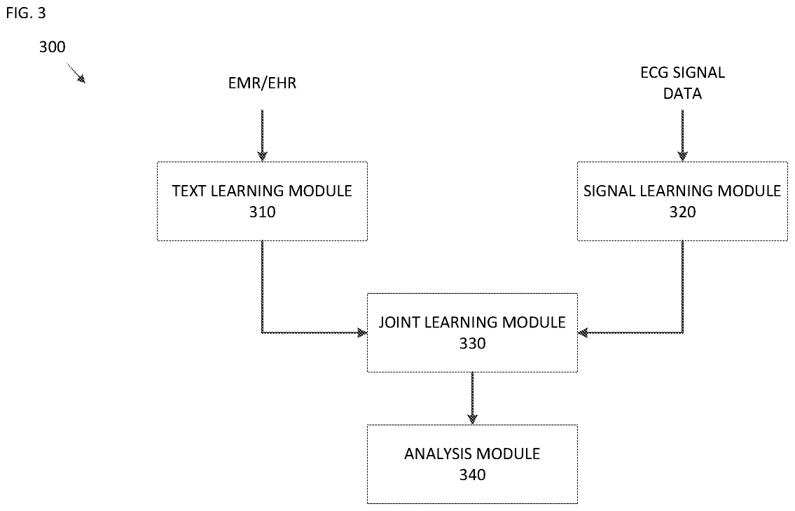

Embodiments described herein include a new model training framework for electromyography (EMG)/ECG analysis, which accepts comprehensive multi-lead ECG signals and adopts geometric properties of electrodes from ECG exams. Specifically, such features are achieved via three techniques: a grouping module, a multi-axis feature extraction module, and a comprehensive task-specific analysis module, as described below with respect to FIG. 3.

Current training frameworks for ECG analysis rely only on an electric signal, which ignores a medical history and a background of a patient. The framework described herein combines advantages from both electric medical records and signal data, to achieve multiple goals of ECG analysis, such as ECG monitoring and alarming and computer-aided diagnosis.

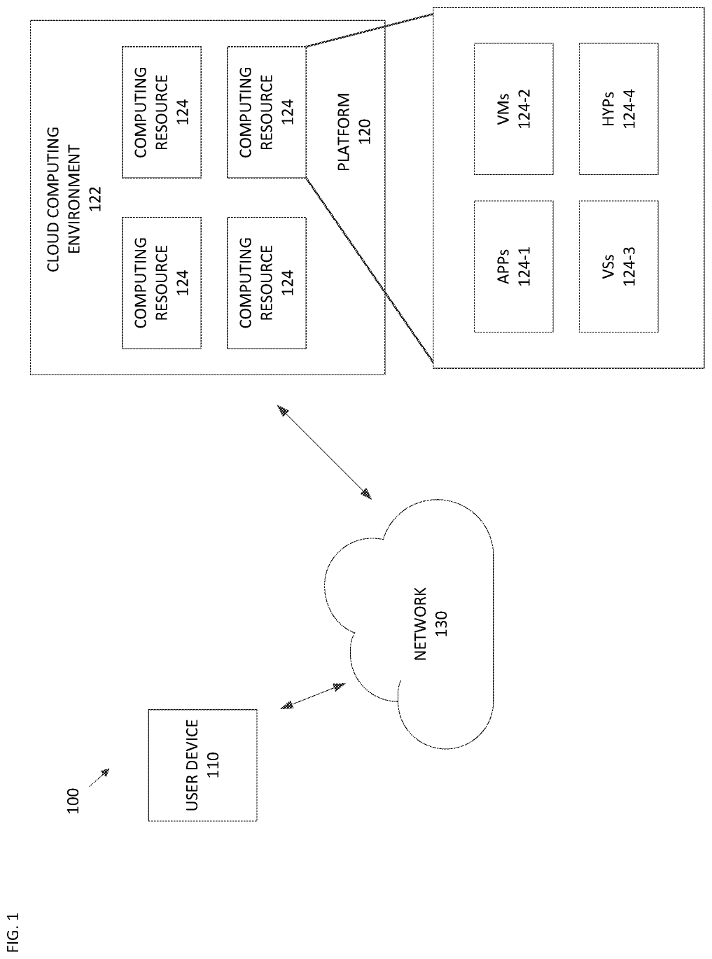

FIG. 1 is a diagram of an environment 100 in which methods, apparatuses and systems described herein may be implemented, according to embodiments. As shown in FIG. 1, environment 100 may include a user device 110, a platform 120, and a network 130. Devices of environment 100 may interconnect via wired connections, wireless connections, or a combination of wired and wireless connections.

User device 110 includes one or more devices capable of receiving, generating, storing, processing, and/or providing information associated with platform 120. For example, user device 110 may include a computing device (e.g., a desktop computer, a laptop computer, a tablet computer, a handheld computer, a smart speaker, a server, etc.), a mobile phone (e.g., a smart phone, a radiotelephone, etc.), a wearable device (e.g., a pair of smart glasses or a smart watch), or a similar device. In some implementations, user device 110 may receive information from and/or transmit information to platform 120.

Platform 120 includes one or more devices as described elsewhere herein. In some implementations, platform 120 may include a cloud server or a group of cloud servers. In some implementations, platform 120 may be designed to be modular such that software components may be swapped in or out depending on a particular need. As such, platform 120 may be easily and/or quickly reconfigured for different uses.

In some implementations, as shown, platform 120 may be hosted in cloud computing environment 122. Notably, while implementations described herein describe platform 120 as being hosted in cloud computing environment 122, in some implementations, platform 120 is not be cloud-based (i.e., may be implemented outside of a cloud computing environment) or may be partially cloud-based.

Cloud computing environment 122 includes an environment that hosts platform 120. Cloud computing environment 122 may provide computation, software, data access, storage, etc. services that do not require end-user (e.g., user device 110) knowledge of a physical location and configuration of system(s) and/or device(s) that hosts platform 120. As shown, cloud computing environment 122 may include a group of computing resources 124 (referred to collectively as “computing resources 124” and individually as “computing resource 124”).

Computing resource 124 includes one or more personal computers, workstation computers, server devices, or other types of computation and/or communication devices. In some implementations, computing resource 124 may host platform 120. The cloud resources may include compute instances executing in computing resource 124, storage devices provided in computing resource 124, data transfer devices provided by computing resource 124, etc. In some implementations, computing resource 124 may communicate with other computing resources 124 via wired connections, wireless connections, or a combination of wired and wireless connections.

As further shown in FIG. 1, computing resource 124 includes a group of cloud resources, such as one or more applications (“APPs”) 124-1, one or more virtual machines (“VMs”) 124-2, virtualized storage (“VSs”) 124-3, one or more hypervisors (“HYPs”) 124-4, or the like.

Application 124-1 includes one or more software applications that may be provided to or accessed by user device 110 and/or platform 120. Application 124-1 may eliminate a need to install and execute the software applications on user device 110. For example, application 124-1 may include software associated with platform 120 and/or any other software capable of being provided via cloud computing environment 122. In some implementations, one application 124-1 may send/receive information to/from one or more other applications 124-1, via virtual machine 124-2.

Virtual machine 124-2 includes a software implementation of a machine (e.g., a computer) that executes programs like a physical machine. Virtual machine 124-2 may be either a system virtual machine or a process virtual machine, depending upon use and degree of correspondence to any real machine by virtual machine 124-2. A system virtual machine may provide a complete system platform that supports execution of a complete operating system (“OS”). A process virtual machine may execute a single program, and may support a single process. In some implementations, virtual machine 124-2 may execute on behalf of a user (e.g., user device 110), and may manage infrastructure of cloud computing environment 122, such as data management, synchronization, or long-duration data transfers.

Virtualized storage 124-3 includes one or more storage systems and/or one or more devices that use virtualization techniques within the storage systems or devices of computing resource 124. In some implementations, within the context of a storage system, types of virtualizations may include block virtualization and file virtualization. Block virtualization may refer to abstraction (or separation) of logical storage from physical storage so that the storage system may be accessed without regard to physical storage or heterogeneous structure. The separation may permit administrators of the storage system flexibility in how the administrators manage storage for end users. File virtualization may eliminate dependencies between data accessed at a file level and a location where files are physically stored. This may enable optimization of storage use, server consolidation, and/or performance of non-disruptive file migrations.

Hypervisor 124-4 may provide hardware virtualization techniques that allow multiple operating systems (e.g., “guest operating systems”) to execute concurrently on a host computer, such as computing resource 124. Hypervisor 124-4 may present a virtual operating platform to the guest operating systems, and may manage the execution of the guest operating systems. Multiple instances of a variety of operating systems may share virtualized hardware resources.

Network 130 includes one or more wired and/or wireless networks. For example, network 130 may include a cellular network (e.g., a fifth generation (5G) network, a long-term evolution (LTE) network, a third generation (3G) network, a code division multiple access (CDMA) network, etc.), a public land mobile network (PLMN), a local area network (LAN), a wide area network (WAN), a metropolitan area network (MAN), a telephone network (e.g., the Public Switched Telephone Network (PSTN)), a private network, an ad hoc network, an intranet, the Internet, a fiber optic-based network, or the like, and/or a combination of these or other types of networks.

The number and arrangement of devices and networks shown in FIG. 1 are provided as an example. In practice, there may be additional devices and/or networks, fewer devices and/or networks, different devices and/or networks, or differently arranged devices and/or networks than those shown in FIG. 1. Furthermore, two or more devices shown in FIG. 1 may be implemented within a single device, or a single device shown in FIG. 1 may be implemented as multiple, distributed devices. Additionally, or alternatively, a set of devices (e.g., one or more devices) of environment 100 may perform one or more functions described as being performed by another set of devices of environment 100.

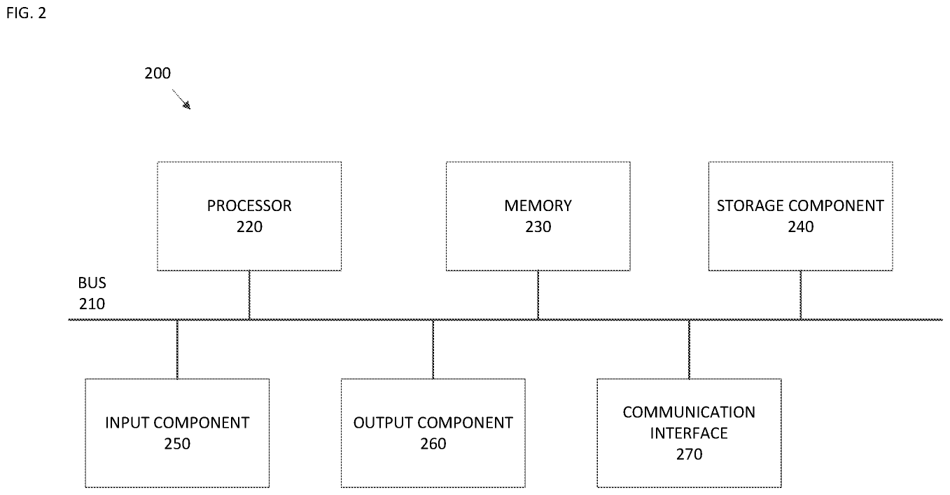

FIG. 2 is a diagram of example components of one or more devices of FIG. 1. A device 200 may correspond to user device 110 and/or platform 120. As shown in FIG. 2, device 200 may include a bus 210, a processor 220, a memory 230, a storage component 240, an input component 250, an output component 260, and a communication interface 270.

Bus 210 includes a component that permits communication among the components of device 200. Processor 220 is implemented in hardware, firmware, or a combination of hardware and software. Processor 220 is a central processing unit (CPU), a graphics processing unit (GPU), an accelerated processing unit (APU), a microprocessor, a microcontroller, a digital signal processor (DSP), a field-programmable gate array (FPGA), an application-specific integrated circuit (ASIC), or another type of processing component. In some implementations, processor 220 includes one or more processors capable of being programmed to perform a function. Memory 230 includes a random access memory (RAM), a read only memory (ROM), and/or another type of dynamic or static storage device (e.g., a flash memory, a magnetic memory, and/or an optical memory) that stores information and/or instructions for use by processor 220.

Storage component 240 stores information and/or software related to the operation and use of device 200. For example, storage component 240 may include a hard disk (e.g., a magnetic disk, an optical disk, a magneto-optic disk, and/or a solid state disk), a compact disc (CD), a digital versatile disc (DVD), a floppy disk, a cartridge, a magnetic tape, and/or another type of non-transitory computer-readable medium, along with a corresponding drive.

Input component 250 includes a component that permits device 200 to receive information, such as via user input (e.g., a touch screen display, a keyboard, a keypad, a mouse, a button, a switch, and/or a microphone). Additionally, or alternatively, input component 250 may include a sensor for sensing information (e.g., a global positioning system (GPS) component, an accelerometer, a gyroscope, and/or an actuator). Output component 260 includes a component that provides output information from device 200 (e.g., a display, a speaker, and/or one or more light-emitting diodes (LEDs)).

Communication interface 270 includes a transceiver-like component (e.g., a transceiver and/or a separate receiver and transmitter) that enables device 200 to communicate with other devices, such as via a wired connection, a wireless connection, or a combination of wired and wireless connections. Communication interface 270 may permit device 200 to receive information from another device and/or provide information to another device. For example, communication interface 270 may include an Ethernet interface, an optical interface, a coaxial interface, an infrared interface, a radio frequency (RF) interface, a universal serial bus (USB) interface, a Wi-Fi interface, a cellular network interface, or the like.

Device 200 may perform one or more processes described herein. Device 200 may perform these processes in response to processor 220 executing software instructions stored by a non-transitory computer-readable medium, such as memory 230 and/or storage component 240. A computer-readable medium is defined herein as a non-transitory memory device. A memory device includes memory space within a single physical storage device or memory space spread across multiple physical storage devices.

Software instructions may be read into memory 230 and/or storage component 240 from another computer-readable medium or from another device via communication interface 270. When executed, software instructions stored in memory 230 and/or storage component 240 may cause processor 220 to perform one or more processes described herein. Additionally, or alternatively, hardwired circuitry may be used in place of or in combination with software instructions to perform one or more processes described herein. Thus, implementations described herein are not limited to any specific combination of hardware circuitry and software.

The number and arrangement of components shown in FIG. 2 are provided as an example. In practice, device 200 may include additional components, fewer components, different components, or differently arranged components than those shown in FIG. 2. Additionally, or alternatively, a set of components (e.g., one or more components) of device 200 may perform one or more functions described as being performed by another set of components of device 200.

Embodiments described herein are designed to achieve multiple data analysis tasks with both medical record text and ECG signal data. A multimodal framework uses a joint learning module that merges information from both the medical record text and ECG signal data simultaneously. The joint learning module adopts individual learning modules for the medical record text and ECG signal data, respectively, and data features from these different data sources are extracted for a final inference module. Therefore, the multimodal framework can be widely applied to various types of analysis tasks. It can also use background knowledge, such as geometric properties and ontologies, for analysis.

FIG. 3 is a diagram of a multimodal framework 300 for heart abnormalities analysis based on an EMR and/or an EHR and ECG signal data of a patient, according to embodiments.

Referring to FIG. 3, the multimodal framework 300 includes a text learning module 310, a signal learning module 320, a joint learning module 330 and an analysis module 340.

The text learning module 310 extracts first informative information from the EMR and/or the EHR of the patient or a user, such as symptoms, a previous history, etc. In detail, the text learning module 310 learns valuable information from the EMR and/or the EHR in a text format. In medical history records of the patient, especially in his or her previous disease history and symptoms, there are some hints for the heart abnormities analysis. Therefore, the text learning module 310 learns this first informative information from the records.

The signal learning module 320 extracts second informative information from the ECG signal data of the patient, the second informative information representing a wave style, signal characteristics, etc. In detail, the signal learning module 320 accepts the pre-processed ECG signal data in different formats (e.g., single-lead, 12-lead) as inputs, and generates feature vectors as outputs.

Model-wise, each of the text learning module 310 and the signal learning module 320 can use any of machine learning approaches such as a support-vector machine (SVM), random forests (RF), or deep learning (DL) models such as a CNN and an RNN. Parameters for each of the text learning module 310 and the signal learning module 320 are trained separately to acquire group-specified extraction approaches.

The joint learning module 330 merges the first and second informative information extracted from both text and signal sides, and generates and outputs one or more specific representations, i.e., one or more feature vectors. In detail, after extracting the first and second informative information from both medical record and ECG signal sides, the joint learning module 330 combines this information together for a final analysis. These combined features can support a final heart abnormalities analysis.

The analysis module 340 performs and finishes a specific task such as clustering, classification, prediction, etc., based on the one or more representations, and then achieves a final goal of the multimodal framework 300. In detail, the analysis module 340 may be an ECG abnormalities analysis module that accepts extracted features and produces final outcomes such as classification results, outlier alarms, and/or predicted diagnosis, based on the one or more representations. A task specific module pool is a collection of different models that are used for various ECG-related tasks. For instance, in the task specific module pool, there may be several statistical process control algorithms for ECG monitoring and alarming, several predictive models and classifier models for computer-aided diagnosis, and some statistical tools for pathological status calculation. Depending on the goal of using the multimodal framework 300, the analysis module 340 deploys an appropriate tool from the pool to finish an end-to-end framework and achieve the final goal.

The training multimodal framework 300 is an end-to-end framework. Compared to existing approaches of an ECG analysis model, the multimodal framework 300 can learn and extract information from both text and signal data to provide a more accurate analysis.

Further, the multimodal framework 300 can accept different types of medical record data (e.g., EMR and EHR) and different types of ECG data (e.g., single-lead, 12-lead) that could provide comprehensive information for better performance of a model.

In embodiments, the text learning module 310 may include any of machine learning algorithms such as an RNN, a CNN or an SVM.

In embodiments, the signal learning module 320 may include any of machine learning algorithms such as an RNN, a CNN or an SVM.

In embodiments, the joint learning module 330 may use a flexible joint strategy, such as a concatenated and weighted combination based on model learning or expert knowledge.

The multimodal framework 300 is designed as an end-to-end procedure in which the whole multimodal framework 300 may be optimized and altered simultaneously. An alternative would be a step-by-step training procedure, in which each of the text learning module 310 and the signal learning module 320 can be trained separately, for instance, using an encoder and decoder structure.

The multimodal framework 300 can be extended to other applications that have heterogamous sources of input.

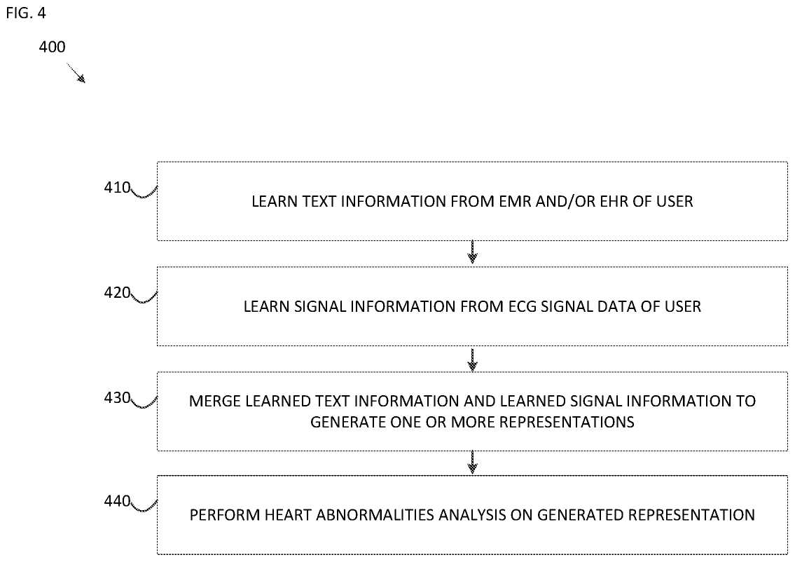

FIG. 4 is a flowchart of a method 400 of performing a heart abnormalities analysis, according to embodiments. In some implementations, one or more process blocks of FIG. 4 may be performed by platform 120. In some implementations, one or more process blocks of FIG. 4 may be performed by another device or a group of devices separate from or including platform 120, such as user device 110.

As shown in FIG. 4, in operation 410, the method 400 includes learning text information from an EMR and/or an EHR of a user.

In operation 420, the method 400 includes learning signal information from ECG signal data of the user.

In operation 430, the method 400 includes merging the learned text information and the learned signal information to generate one or more representations of the text information and the signal information that are merged.

In operation 440, the method 400 includes performing the heart abnormalities analysis on the generated one or more representations.

The ECG signal data may include either one or both of single-lead ECG signal data and 12-lead ECG signal data.

The signal information may include one or more feature vectors representing a wave style and/or one or more signal characteristics.

Each of the learning of the text information and the learning of the signal information may include generating a respective one of the text information and the signal information that includes one or more feature vectors, using any one or any combination of an SVM, RFs, and DL models including a CNN and an RNN.

The merging of the learned text information and the learned signal information may include generating the one or more representations including one or more feature vectors, using a concatenated and weighted combination based on model learning or expert knowledge.

The performing the heart abnormalities analysis may include performing any one or any combination of clustering the generated one or more representations, classification of the generated one or more representations, prediction of a diagnosis, based on the generated one or more representations, and generating an outlier alarm, based on the generated one or more representations.

The learning of the text information, the learning of the signal information, the merging of the learned text information and the learned signal information and the performing the heart abnormalities analysis may be performed simultaneously.

Although FIG. 4 shows example blocks of the method 400, in some implementations, the method 400 may include additional blocks, fewer blocks, different blocks, or differently arranged blocks than those depicted in FIG. 4. Additionally, or alternatively, two or more of the blocks of the method 400 may be performed in parallel.

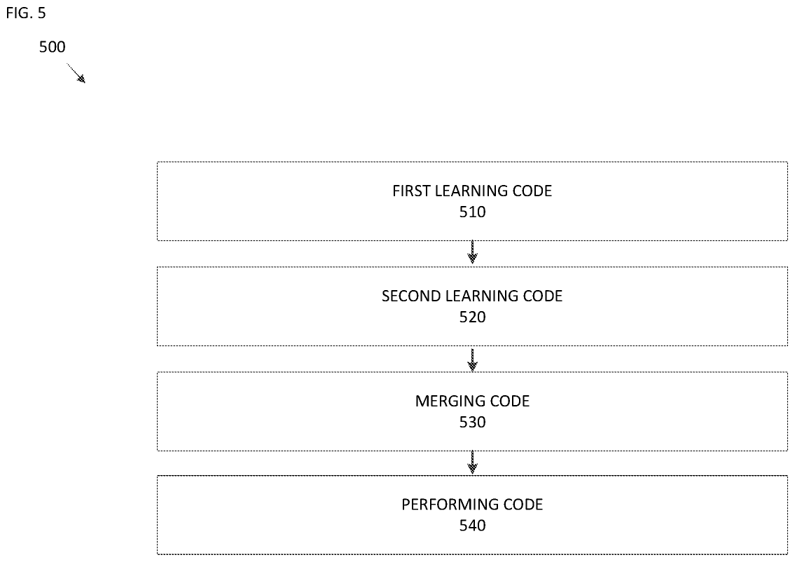

FIG. 5 is a diagram of an apparatus 500 performing a heart abnormalities analysis, according to embodiments. As shown in FIG. 5, the apparatus 500 includes first learning code 510, second learning code 520, merging code 530 and performing code 540.

The first learning code 510 is configured to learn text information from an EMR and/or an EHR of a user.

The second learning code 520 is configured to learn signal information from ECG signal data of the user.

The merging code 530 is configured to merge the learned text information and the learned signal information to generate a representation of the text information and the signal information that are merged.

The performing code 540 is configured to perform the heart abnormalities analysis on the generated representation.

The ECG signal data may include either one or both of single-lead ECG signal data and 12-lead ECG signal data.

The signal information may include one or more feature vectors representing a wave style and/or one or more signal characteristics.

Each of the first learning code and the second learning code may be further configured to generate a respective one of the text information and the signal information that includes one or more feature vectors, using any one or any combination of an SVM, RFs, and DL models including a CNN and an RNN.

The merging code may be further configured to generate the one or more representations including one or more feature vectors, using a concatenated and weighted combination based on model learning or expert knowledge.

The performing code may be further configured to perform any one or any combination of clustering the generated one or more representations, classification of the generated one or more representations, prediction of a diagnosis, based on the generated one or more representations, and generating an outlier alarm, based on the generated one or more representations.

The foregoing disclosure provides illustration and description, but is not intended to be exhaustive or to limit the implementations to the precise form disclosed. Modifications and variations are possible in light of the above disclosure or may be acquired from practice of the implementations.

As used herein, the term component is intended to be broadly construed as hardware, firmware, or a combination of hardware and software.

It will be apparent that systems and/or methods, described herein, may be implemented in different forms of hardware, firmware, or a combination of hardware and software. The actual specialized control hardware or software code used to implement these systems and/or methods is not limiting of the implementations. Thus, the operation and behavior of the systems and/or methods were described herein without reference to specific software code—it being understood that software and hardware may be designed to implement the systems and/or methods based on the description herein.

Even though combinations of features are recited in the claims and/or disclosed in the specification, these combinations are not intended to limit the disclosure of possible implementations. In fact, many of these features may be combined in ways not specifically recited in the claims and/or disclosed in the specification. Although each dependent claim listed below may directly depend on only one claim, the disclosure of possible implementations includes each dependent claim in combination with every other claim in the claim set.

No element, act, or instruction used herein should be construed as critical or essential unless explicitly described as such. Also, as used herein, the articles “a” and “an” are intended to include one or more items, and may be used interchangeably with “one or more.” Furthermore, as used herein, the term “set” is intended to include one or more items (e.g., related items, unrelated items, a combination of related and unrelated items, etc.), and may be used interchangeably with “one or more.” Where only one item is intended, the term “one” or similar language is used. Also, as used herein, the terms “has,” “have,” “having,” or the like are intended to be open-ended terms. Further, the phrase “based on” is intended to mean “based, at least in part, on” unless explicitly stated otherwise.

What is claimed is:

1. A method of performing a heart abnormalities analysis, the method comprising:
learning text information from an electronic medical record (EMR) and/or an electronic health record (EHR) of a user;
learning signal information from electrocardiography (ECG) signal data of the user;
merging the learned text information and the learned signal information to generate one or more representations of the text information and the signal information that are merged; and
performing the heart abnormalities analysis on the generated one or more representations.

2. The method of claim 1, wherein the ECG signal data comprises either one or both of single-lead ECG signal data and 12-lead ECG signal data.

3. The method of claim 1, wherein the signal information comprises one or more feature vectors representing a wave style and/or one or more signal characteristics.

4. The method of claim 1, wherein each of the learning of the text information and the learning of the signal information comprises generating a respective one of the text information and the signal information that comprises one or more feature vectors, using any one or any combination of a support-vector machine (SVM), random forests (RF), and deep learning (DL) models including a convolutional neural network (CNN) and a recurrent neural network (RNN).

5. The method of claim 1, wherein the merging of the learned text information and the learned signal information comprises generating the one or more representations comprising one or more feature vectors, using a concatenated and weighted combination based on model learning or expert knowledge.

6. The method of claim 1, wherein the performing the heart abnormalities analysis comprises performing any one or any combination of clustering the generated one or more representations, classification of the generated one or more representations, prediction of a diagnosis, based on the generated one or more representations, and generating an outlier alarm, based on the generated one or more representations.

7. The method of claim 1, wherein the learning of the text information, the learning of the signal information, the merging of the learned text information and the learned signal information and the performing the heart abnormalities analysis are performed simultaneously.

8. An apparatus for performing a heart abnormalities analysis, the apparatus comprising:
at least one memory configured to store program code; and
at least one processor configured to read the program code and operate as instructed by the program code, the program code including:
first learning code configured to cause the at least one processor to learn text information from an electronic medical record (EMR) and/or an electronic health record (EHR) of a user;
second learning code configured to cause the at least one processor to learn signal information from electrocardiography (ECG) signal data of the user;
merging code configured to cause the at least one processor to merge the learned text information and the learned signal information to generate a representation of the text information and the signal information that are merged; and
performing code configured to cause the at least one processor to perform the heart abnormalities analysis on the generated representation.

9. The apparatus of claim 8, wherein the ECG signal data comprises either one or both of single-lead ECG signal data and 12-lead ECG signal data.

10. The apparatus of claim 8, wherein the signal information comprises one or more feature vectors representing a wave style and/or one or more signal characteristics.

11. The apparatus of claim 8, wherein each of the first learning code and the second learning code is further configured to cause the at least one processor to generate a respective one of the text information and the signal information that comprises one or more feature vectors, using any one or any combination of a support-vector machine (SVM), random forests (RF), and deep learning (DL) models including a convolutional neural network (CNN) and a recurrent neural network (RNN).

12. The apparatus of claim 8, wherein the merging code is further configured to cause the at least one processor to generate the one or more representations comprising one or more feature vectors, using a concatenated and weighted combination based on model learning or expert knowledge.

13. The apparatus of claim 8, wherein the performing code is further configured to cause the at least one processor to perform any one or any combination of clustering the generated one or more representations, classification of the generated one or more representations, prediction of a diagnosis, based on the generated one or more representations, and generating an outlier alarm, based on the generated one or more representations.

14. A non-transitory computer-readable medium storing instructions that, when executed by at least one processor of a device, cause the at least one processor to:
learn text information from an electronic medical record (EMR) and/or an electronic health record (EHR) of a user;
learn signal information from electrocardiography (ECG) signal data of the user;
merge the learned text information and the learned signal information to generate a representation of the text information and the signal information that are merged; and
perform a heart abnormalities analysis on the generated representation.

15. The non-transitory computer-readable medium of claim 14, wherein the ECG signal data comprises either one or both of single-lead ECG signal data and 12-lead ECG signal data.

16. The non-transitory computer-readable medium of claim 14, wherein the signal information comprises one or more feature vectors representing a wave style and/or one or more signal characteristics.

17. The non-transitory computer-readable medium of claim 14, wherein the instructions further cause the at least one processor to generate a respective one of the text information and the signal information that comprises one or more feature vectors, using any one or any combination of a support-vector machine (SVM), random forests (RF), and deep learning (DL) models including a convolutional neural network (CNN) and a recurrent neural network (RNN).

18. The non-transitory computer-readable medium of claim 14, wherein the instructions further cause the at least one processor to generate the one or more representations comprising one or more feature vectors, using a concatenated and weighted combination based on model learning or expert knowledge.

19. The non-transitory computer-readable medium of claim 14, wherein the instructions further cause the at least one processor to perform any one or any combination of clustering the generated one or more representations, performing classification on the generated one or more representations, performing prediction of a diagnosis, based on the generated one or more representations, and generating an outlier alarm, based on the generated one or more representations.

20. The non-transitory computer-readable medium of claim 14, wherein the instructions further cause the at least one processor to simultaneously learn the text information, learn the signal information, merge the learned text information and the learned signal information, and perform the heart abnormalities analysis.
